(19) United States
(12) Patent Application Publication
(10) Pub. No.: US 2009/0306015 A1
(43) Pub. Date: Dec. 10, 2009

(54) PHARMACEUTICAL COMPOSITIONS AND METHODS OF USE OF HIGHLY LIPOPHILIC SULFHYDRYL COMPOUNDS

(75) Inventors: Stephen Gately, Scottsdale, AZ (US); Robert West, Madison, WI (US)

Correspondence Address: K&L Gates LLP, P.O. Box 1135, CHICAGO, IL, 60690, US

(73) Assignee: Silamed, Inc., Scottsdale, AZ (US)

(21) Appl. No.: 12/158,249

(22) PCT Filed: Dec. 20, 2006
(86) PCT No.: PCT/US06/62418

§ 371 Date: Oct. 30, 2008

Related U.S. Application Data
(60) Provisional application No. 60/752,278, filed on Dec. 20, 2005.

(51) Int. Cl. A61K 31/695; C07F 7/10; A61P 9/00; A61P 35/00; A61P 25/00

(52) U.S. Cl. 514/63; 556/419

(57) ABSTRACT

Novel compositions of silicon-containing sulfhydryl compounds, their preparation and use in methods for treating disease are described. Silicon confers lipophilicity that can enhance the penetration of the silicon derivative sulfhydryl compounds across the gut wall, cell membranes and blood brain barrier, thus improving therapeutic properties including bioavailability, metabolism, and/or pharmacokinetics. The organosilyl group provides compounds having improved pharmacokinetics. The invention encompasses novel compounds, analogs, prodrugs and pharmaceutically acceptable salts thereof, pharmaceutical compositions and methods for treatment of diseases and other maladies or conditions and the like. The subject invention also relates to processes for making such compounds as well as to intermediates useful in such processes.

### BACKGROUND OF THE INVENTION

The present invention relates to lipophilic antioxidant compounds, pharmaceutical compositions containing same and their use for preventing or reducing oxidative stress. More particularly, the present invention relates to novel non-central nervous system (CNS) and CNS targeted antioxidants and their use in treating non-CNS and CNS disorders, diseases or conditions associated with a formation of oxidative stress.

### Oxidative Stress

The cellular physiological reduction-oxidation (redox) state, which is dependent on concentrations of oxygen and reactive oxygen species (ROS), is involved in controlling central biochemical regulatory processes, such as tyrosine phosphorylation, regulation of transcription and alteration in messenger RNA stability and it is finely balanced by specific enzymes, such as superoxide dismutase (SOD), catalase, gluthatione peroxidase and thioredoxin, and selective antioxidants, such as glutathione. Regulated homeostasis of the intracellular redox state is essential to the proper physiological functioning of the cell, however, overproduction of (ROS), at levels exceeding the neutralization capacity of cellular antioxidant defenses, generates an oxidative state, termed oxidative stress. Such oxidative stress can lead to oxidative injury via processes such as inflammation, apoptosis and mutagenesis.

Inflammation, a normal physiological process involving limited tissue injury, can be pathogenic if uncontrolled, as under conditions of excessive oxidative stress. In such cases, elevation of ROS, via alterations in expression of redox state-responsive genes, causes the ubiquination and destruction of the NF-kappa B inhibitory proteins, thereby allowing NF-kappa B to bind to target gene promoters, a pivotal event in the upregulation of multiple pro-inflammatory cytokines. An excess of free radicals has been identified in many diseases associated with inflammation, such as sepsis, multiple sclerosis (MS), stroke, myocarditis and rheumatoid arthritis.

While the development and maintenance of a healthy tissue involves properly regulated apoptosis, interference with this process contributes to various pathologies including tumor promotion, immunodeficiency diseases and neurodegenerative disorders. It has been shown that elevation of the intracellular oxidative state, either via addition of reactive oxygen species (ROS) or depletion of cellular antioxidants, can cause apoptosis and much evidence has accumulated linking oxidative stress to activation of specific enzymes involved in apoptosis.

One such enzyme, essential in the signaling pathway of cytochrome c mediated apoptosis, is c-Jun N-terminal kinase (JNK) that is activated in response to UV radiation, cisplatinum treatment or cellular stress. It has been demonstrated that disruption of JNK protects against UV induced apoptosis, resulting in impairment of the mitochondrial death-signaling pathway.

ROS have been shown to play a role as intermediate factors in the pathway of various signal transduction pathways involving thioredoxin, a ubiquitous enzyme in all living cells containing a specific redox-active site. Thioredoxin acts as an inhibitor of oxidative stress induced apoptosis by binding to, and thereby inhibiting, apoptosis signal regulating kinase-1 (ASK1), a protein mediating oxidative stress-induced apoptosis via a redox state responsive domain. However, under conditions of excessive oxidative stress, oxidized thioredoxin dissociates from ASK1, thereby activating it and triggering apoptosis.

Pathologies Associated with Oxidative Stress

Oxidant injury has been implicated in the pathology of a wide-ranging variety of diseases, including many of major clinical and economic impact, such as cardiovascular, neurological, metabolic, infectious, hepatic, pancreatic, rheumatoid, malignant and immunological diseases, as well as conditions such as sepsis, cataract, amyotrophic lateral sclerosis and congenital diseases such as Down's syndrome, multiple organ dysfunction and cystic fibrosis.

Described below are some of the most widespread and devastating diseases in which oxidative stress is an etiological factor.

Neurodegenerative pathologies: involvement of inflammation and oxidative stress: Evidence has accumulated demonstrating a strong linkage of oxidative stress with pathogenesis of major human neurodegenerative disorders including Parkinson's disease, Alzheimer's disease, Creutzfeldt-Jakob disease as well as MS.

The different pathological markers characteristic of various neurodegenerative diseases, such as Lewy bodies in Parkinson's disease and amyloid plaques in Alzheimer's disease, indicate different causal factors in the initiation of these diseases. However, there is growing evidence that, once initiated, the progression of a large number of neurodegenerative diseases follows similar cellular pathways. Namely, elevation of the intracellular oxidative state in specific regions of the CNS appears to be an important factor in the etiology of diseases such as Alzheimer's disease, Parkinson's disease, spongiform encephalopathies, degenerative diseases of the basal ganglia, motoneuron diseases and memory loss.

For example, a role for oxidative stress in the pathogenesis of Alzheimer's disease was indicated in a recent analysis of the relationship between beta-amyloid protein fragment and oxygen radical formation. This study employed a highly sensitive system, utilizing monitoring blood vessel vasoactive responses, in which beta-amyloid-mediated enhancement of phenylephrine-mediated vasoconstriction could be abrogated by pretreatment of blood vessels with SOD, an enzyme which scavenges oxygen free radicals. Other studies have shown that oxidative stress and free radical production are linked to the presence of beta-amyloid fragment (amino acids 25-35) and likely contribute to neurodegenerative events associated with Alzheimer's disease. Further studies have shown extensive RNA oxidation in neurons in Alzheimer's disease and Down's syndrome and genetic evidence for oxidative stress in Alzheimer's disease has also been reported.

Evidence of a role for elevated oxidative stress in pathogenesis of MS was provided in studies analyzing the role of metallothioneins, enzymes involved in maintenance of redox homeostasis, in MS or experimental autoimmune encephalomyelitis (EAE), in studies demonstrating increased lipid peroxidation in serum and cerebrospinal fluid of MS patients and in studies demonstrating the role of heme oxygenase-1 (HO-1), a heat shock protein induced by oxidative stress, in the progression of EAE.

In the case of scrapies, a type of spongiform encephalopathy occurring in sheep, it was demonstrated that pathogenesis is mediated via microglia cells that respond to prion protein fragment PrP¹⁰⁶⁻¹²⁶ by increasing oxygen radical production.

Diabetes: There is convincing experimental and clinical evidence that the generation of ROS is increased in both types of diabetes and that the onset of diabetes is closely associated with oxidative stress. Recently, it was demonstrated that intracellular content of the oxidant H₂O₂, visualized with 2′,7′-dichlorofluorescein and quantified by flow cytometry, is increased following treatment with high glucose levels. Concomitant elevation of lactate dehydrogenase activity was detected suggesting that high glucose promotes necrotic cell death through H₂O₂ formation, which may contribute to the development of diabetic vasculopathy. Consistent with these results, a recent study has demonstrated that long-term administration of antioxidants can inhibit the development of the early stages of diabetic retinopathy. Other studies carried out with treatment of diabetic rats with antioxidants suggest that diabetes-induced oxidative stress and the generation of superoxide may be partially responsible for the development of diabetic vascular and neural complications.

Cataract formation: A role for oxidant injury in cataract formation was shown in early studies demonstrating that decreased levels of the antioxidant hepatic glutathione-S-transferase (GSH) are associated with increasing opacity of the lens. Later studies have shown that in the mammalian lens, intracellular oxidants produced by light induced oxidative processes cause oxidative damage, result in changes in gene expression, and are causally related to cataract formation. It is presently believed that H₂O₂ is the major oxidant to which the lens is exposed.

Infectious diseases. Harmful levels of oxygen free radicals and nitric oxide (NO) are generated in a diverse range of, and are essential to, the pathogenesis of many types of microbial infections. Viral diseases whose pathogenesis is associated with oxidative stress include hepatitis C, AIDS, influenza and diseases caused by various neurotropic agents. In many kinds of viral infections high levels of NO generate highly reactive nitrogen oxide species including reactive oxygen intermediates as well as peroxynitrite, via interaction with oxygen radicals. These species of reactive nitrogen cause oxidant injury as well as mutagenesis via oxidation of various biomolecules. Recent evidence has also demonstrated that oxidative stress induced by NO causes further harm by increasing viral mutation rates and by suppressing type 1 helper T cell function. For example, studies employing the equine influenza virus (EIV) influenza model have shown that viral infection causes cytopathogenic effects and apoptosis as a result of oxidative stress. Another study has shown that progression of human hepatitis C virus infection involves triggering of oxidative stress via a mechanism in which the non-structural HCV protein NS5A triggers elevation of ROS in mitochondria, leading to the nuclear translocation and constitutive activation of the pro-inflammatory transcription factors NF-kappa B and STAT-3.

Neurological dysfunction following cardiac surgery: Cardiac operations, such as coronary bypass surgery, following multiple infarctions has been shown to significantly increase the risk of neurologic dysfunction, such as impairment of brain function and memory. Studies have provided evidence that such neurological impairment is associated with oxidative stress.

Cardiovascular diseases: The pathogenesis of major cardiovascular diseases, such as atherosclerosis, hypertension, stroke and restenosis, has been shown to involve oxidative stress. Such oxidant stress in the vasculature causes adverse vessel reactivity, vascular smooth muscle cell proliferation, macrophage adhesion, platelet activation, and lipid peroxidation. In the case of atherosclerosis, one of the leading causes of mortality in the developed world, pathogenesis specifically involves inflammation and oxidation of lipoprotein-derived lipids.

Recent studies have shown that cerebral ischemia followed by reperfusion leads to elevated oxidative stress and that such oxidative stress can cause damage to genes in brain tissue despite functional DNA repair mechanisms. Involvement of such oxidative stress in ischemia-associated pathogenesis was further demonstrated in studies reporting increased infarct size and exacerbated apoptosis in glutathione peroxidase-1 (Gpx-1) knockout mouse brain subjected to ischemia/reperfusion injury.

Cancer: Studies have shown that oxidative stress/reactive oxygen species are involved in development of some cancers. Additionally, cancer patients treated with various chemotherapeutic agents often complain of forgetfulness, lack of concentration, dizziness (collectively called chemobrain, chemo-fog, or chemotherapy-related cognitive dysfunction). Mild cognitive impairment consistent with chemobrain caused by the anti-cancer drug adriamycin is reported to be related to free radical mediated oxidative stress.

Thus, the pathogenesis of a very broad variety of diseases involves oxidative stress and, as such, methods of reducing oxidative state may provide an attractive means of treating such diseases.

### Methods of Treating Disease Via Reduction of Oxidative Stress

Various prior art methods of treating diseases associated with oxidative stress via reduction of oxidative stress have been attempted and have demonstrated the potential effectiveness of treating disease by restoring redox balance. These have involved either prevention of enzymatic production of ROS by specific inhibitors or introduction of exogenous antioxidants for restoring redox balance.

Diseases of the CNS: To overcome high oxidative stress for the treatment of diseases of the CNS, it is desirable to administer agents capable of reducing oxidative stress into the CNS. However, the CNS is physiologically separated from the rest of the body and from the peripheral blood circulation, by the blood brain barrier (BBB). Since the BBB constitutes a very effective barrier for the passage of agents, such as antioxidants, lacking a selective transporter, such as enzymes or other proteins capable of decreasing oxidative stress, administration of such agents must be via direct injection into the brain or cerebrospinal fluid (CSF). Such a route of administration, however, is unacceptably risky, cumbersome and invasive and thus represents a major drawback for this treatment modality.

One approach has employed administration of the antioxidants vitamin E and vitamin C for treatment of neurological diseases, such as Parkinson's disease. Vitamin E was found to be ineffective at decreasing oxidative stress in the substantia nigra and, although capable of crossing the BBB, is trapped in the cell membrane and therefore does not reach the cytoplasm where its antioxidant properties are needed. Vitamin C was shown to cross the BBB to some extent, via a selective transporter, nevertheless it has also been shown to be ineffective in treating neurodegenerative diseases of the CNS.

In another approach, antioxidant compounds characterized by a combination of low molecular weight and membrane miscibility properties for permitting the compounds to cross the BBB of an organism, a readily oxidizable (i.e., reducing) chemical group for exerting antioxidation properties and a chemical make-up for permitting the compounds or their intracellular derivative to accumulate within the cytoplasm of cells, have been employed to treat pathology, including CNS pathology, associated with oxidative stress.

Diseases of Non-CNS Tissues

Systemic administration of antioxidants: The major prior art approach used for reducing oxidative stress in non-CNS tissues has employed administration antioxidants.

The antioxidant n-acetylcysteine (NAC) has been employed to treat canine kidney cells so as to attenuate EIV-induced cytopathic effect and apoptosis and to treat atherosclerosis and restenosis following angioplasty. Dimers of NAC have also been employed for treating atherosclerosis.

The sulphur-containing fatty acid with antioxidant properties, tetradecylthioacetic acid, has been employed to achieve long-term reduction of restenosis following balloon angioplasty in porcine coronary arteries.

The antioxidants pyrrolidine dithiocarbamate (PDTC) and NAC have been used to prevent pathogenic HCV mediated constitutive activation of the pro-inflammatory transcription factor STAT-3.

Synthetic antioxidants have also been employed to treat oxidative stress related disease. For example, treatment of asthma has been attempted by reducing the levels of free oxygen using the synthetic reactive oxygen inhibitor 2,4-diaminopyrrolo-2,3-dipyrimidine.

Apoptosis in an ischemic swine heart model has been treated with ebselen, a glutathione peroxidase mimic.

The cytosolic antioxidant, copper/zinc superoxide dismutase, has been employed to treat blood-brain barrier disruption and infarction following cerebral ischemia-reperfusion. Attenuation of ischemia-induced mouse brain injury has been attempted by administration of SAG, a redox-inducible antioxidant protein.

Administration of metabolic regulators of antioxidants: Another approach has attempted to employ metabolic regulators of antioxidants to reduce oxidative stress. One study has attempted prevention of cataract in a chick embryo model via administration of thyroxine to drive metabolic maintenance of hepatic GSH levels so as to reduce oxidative stress induced by glucocorticoids.

Hemin, an inducer of the oxidative stress induced protein, heme oxygenase-1, has been utilized to inhibit progression of EAE.

Administration of corticosteroids has been employed to treat lipid peroxidation in MS patients.

Stimulation of production of the endogenous antioxidant reduced glutathione has been attempted for treating acute respiratory distress syndrome (ARDS), a condition characterized by overproduction of oxidants or ROS by the immune system, by administration of the drug pro-cysteine (Free Radical Sciences Inc., CA, U.S.). This drug functions by boosting cellular production of glutathione by upregulation of cellular cysteine uptake.

A common feature characterizing all of the above described and other antioxidant compounds is their limited diversity in structure, body distribution, cellular distribution, organelle distribution, and/or antioxidant properties, etc. As such, any given antioxidant may prove useful for some applications, yet less or non-useful for other applications. In some cases, a specific antioxidant may efficiently reduce oxidative stress in some body parts, some cells, or some subcellular structures, yet not in others.

There is thus, a great need for, and it would be highly advantageous to have, a lipophilic, poorly water soluble antioxidant compound to combat disease, syndromes and conditions associated with formation of oxidative stress, both in non-CNS and CNS tissues.

### SUMMARY OF THE INVENTION

The present invention provides novel sulfhydryl compounds having improved therapeutic properties, including pharmacokinetic properties. Methods for preparing and using these compounds are also disclosed. The invention covers drugs containing silicon that have beneficial properties. The approach involves inserting silicon atom(s), and selecting those modified drugs having improved biological or therapeutic properties. A review of silicon chemistry is provided in Tacke and Zilch, Endeavour, New Series, 10, 191-197 (1986); and Showell, G A and Mills, J S, Chemistry challenges in lead optimization: silicon isosteres in drug discovery. Drug Discovery Today 8(12): 551-556, 2003.

The compositions of the invention include carboxylate containing drugs, including COOH-containing drugs, such as COOH-containing sulfhydryl derivatives that exhibit the improved biological properties and improved pharmacokinetics. These molecules retain the antioxidation properties of their unmodified counterpart parent drugs, and yet exhibit other effects not exhibited by the drug prior to derivatization.

Silicon-containing sulfhydryl compounds of the invention include but are not limited to N-acetylcysteine, D-penicillamine, L-penicillamine, a mixture of D-penicillamine and L-penicillamine, N-Acetyl-D-penicillamine, N-Acetyl-DL-penicillamine, captopril (N—[(S)-3-Mercapto-2-methylpropionyl]-L-proline), D-methionine, L-methionine, a mixture of D-methionine and L-methionine, homomethionine, S-adenosyl-L-methionine, ethionine, aurothiomalate ((1,2-Dicarboxyethylthio)gold).

One object of the present invention is to generate lipophilic silicon analogs of the carboxylic acid moiety of certain sulfhydryl compounds. The resulting compositions are also covered by the invention.

Methods are also provided for administering to a mammal, particularly a human, a treatment-effective amount of a silicon-containing sulfhydryl derivative. In an embodiment the sulfhydryl compound contains a carboxylic acid moiety and includes pharmaceutically acceptable salts thereof. Preferably, the compound is a sulfhydryl-containing drug, or a pharmaceutically acceptable salt thereof.

It is a further object of the present invention to provide compounds that demonstrate enhanced pharmacokinetics, and/or altered metabolism and/or improved drug bioavailability and half-life as compared to underivatized counterparts.

It is also an object of the present invention to provide compounds that demonstrate enhanced lipophilicity, improved gastrointestinal absorption, and enhanced oral bioavailability. It is still a further object of the present invention that the novel compounds have an improved pharmacological profile compared to the underivatized counterpart compounds, and as a result, are better tolerated by humans or animals.

Pharmaceutical composition comprising one or more of the compounds as described herein in a pharmaceutically acceptable diluent or carrier are also contemplated. Many pharmaceutical diluents are known and can be used. It is well within the skill of a person having skill in the pharmaceutical formulation arts to provide such compositions.

Compounds of the invention also include diasteriomers, racemates, isolated enantiomers, the compounds can be in hydrated forms, solvated forms, various crystalline forms or amorphous forms, as are known. Some of the crystalline forms of the compounds may exist in more than one polymorphic form and as such all forms are intended to be included in the present invention. Amorphous solids, in contrast to crystalline forms, do not possess a distinguishable crystal lattice and do not have an orderly arrangement of structural units. Amorphous forms are generally more soluble, and thus they can be desirable for pharmaceutical purposes because the bioavailability of amorphous compounds may be greater than their crystalline counterparts. Certain methods for generating these forms are known and it is within the skill of one having skill in the art to produce them using such methods.

Additional features and advantages of the present invention are described in, and will be apparent from, the following Detailed Description of the Invention and the figures.

### DETAILED DESCRIPTION OF THE INVENTION

In an embodiment, the invention is directed to a particular class of compounds having one or more silicon atoms. Silicon is highly lipophilic and thus enhances the penetration of the compounds across the gut wall, cell membranes and blood brain barrier.

The present invention provides compounds incorporating silicon atom(s) that demonstrate enhanced pharmaceutical properties.

Specifically, preferred silicon-containing sulfhydryl derivatives useful in the present invention include but are not limited to N-acetylcysteine, D-penicillamine, L-penicillamine, a mixture of D-penicillamine and L-penicillamine, N-Acetyl-D-penicillamine, N-Acetyl-DL-penicillamine, captopril (N—[(S)-3-Mercapto-2-methylpropionyl]-L-proline), D-methionine, L-methionine, a mixture of D-methionine and L-methionine, homomethionine, S-adenosyl-L-methionine, ethionine, aurothiomalate ((1,2-Dicarboxyethylthio)gold).

In one embodiment, an agent described above comprises a compound having the structural formula (I)

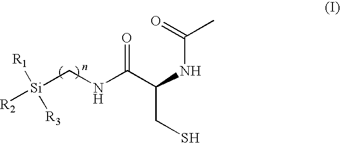

n can be any integral valve that produces an active compound, preferably 1-6;

R₁, R₂, R₃ can be any group that does not substantially interfere with compound formation. Each R can be the same or different and can include, by way of example, methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and the like; or a pharmaceutically acceptable salt thereof.

In another embodiment, the invention relates to a pharmaceutical composition for preventing or reducing oxidative stress, the composition comprising a pharmaceutically acceptable carrier and, an effective amount of at least one compound of formula (I). The pharmaceutical composition has excellent pharmacokinetic profiles for treating mammals, particularly humans with high safety margin.

In yet another embodiment, the invention relates to processes for producing derivatives of formula (I) that can be obtained by reacting a compound of formula (II) with a compound of formula (III) to generate stable silyl compounds of formula (I).

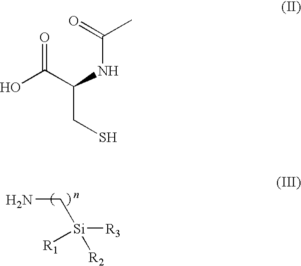

Preferred silicon derivatives of formula (III) include but are not limited to, aminomethyltrimethylsilane, aminopropyltrimethylsilane, (dimethyl(propyl)silyl)methanamine, aminobutyltrimethylsilane, (butyldimethylsilyl)methanamine, aminopentyltrimethylsilane, (dimethyl(pentyl)silyl)methanamine, aminohexyltrimethylsilane, (dimethyl(hexyl)silyl)methanamine, aminoheptyltrimethylsilane, (dimethyl(heptyl)silyl)methanamine, 1,1-dimethylsilinan-3-amine, 4-trimethylsilylaniline, (4-trimethylsilyl)phenyl)methanamine, 4-((trimethylsilyl)methyl)benzamine, 2-trimethylsilyl-5-aminopyridine, (dimethyl(pyridin-3-yl)silyl)methanamine, 2-(dimethyl(pyridin-3-yl)silyl)ethanamine, (dimethyl(phenyl)silyl)-methanamine, ((4-fluorophenyl)dimethylsilyl)methanamine, ((4-chlorophenyl)dimethylsilyl)methanamine, ((4-methoxyphenyl)dimethylsilyl)methanamine, (dimethyl(phenyl)silyl)-ethanamine, ((4-fluorophenyl)dimethylsilyl)ethanamine, ((4-chlorophenyl)dimethylsilyl)ethanamine, ((4-methoxyphenyl)dimethylsilyl)ethanamine.

Further scope of the applicability of the present invention will become apparent from the detailed description provided below. However, it should be understood that the following detailed description and examples, while indicating certain embodiments of the invention, are given by way of illustration only since various changes and modifications within the spirit and scope of the invention will become apparent to those skilled in the art from this detailed description.

The above and other objects, features, and advantages of the present invention will be better understood from the following detailed description all of which are given by way of illustration only, and are not limitative of the present invention.

### Compound Preparation

### General Procedure to Prepare Silyl Amines

Many methods are known for preparing the substituted silyl amines of the invention and can be used. Any mixtures of final products or intermediates obtained can be separated on the basis of the physical-chemical differences of the constituents, in known manner, into the pure final products or intermediates, for example by chromatography, distillation, fractional crystallization, or by formation of a salt as appropriate in the circumstances.

The following routes of synthesis are merely exemplary methods for preparing formula III compounds.

The procedures can utilize silyl derivatives obtained from tetrachlorosilane (SiCl₄) or trichlorochlormethyl silane (Cl₃SiCH₂Cl) or a tailored modification thereof as starting materials. In essence these silyl derivatives are of the formulae VI and VII with R₄, R₅ and R₆ as defined for formula II or they are in a reaction-protected form of the R₄, R₅ and R₆ substituents.

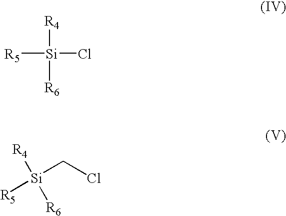

One method for the preparation of the R₄, R₅ and R₆ substituted silanes of formulae IV and V is successive alkylations of tetrachloro-silane and trichlorochloromethyl silane using organo-magnesium halide derivatives of the appropriate R₄, R₅ and R₆ substituents. For example, SiCl₄ is reacted with R₄ Mg halides to produce R₄ SiCl₃ compounds that are reacted with R₅ Mg halides to produce R₄ R₅ SiCl₂ compounds that are reacted with R₆ Mg halides to produce R₄, R₅ and R₆ SiCl compounds. Analogously, R₄ Si(Cl₂)CH₂Cl, R₄ R₅ Si(Cl)CH₂Cl and R₄ R₅ R₆ SiCH₂Cl compounds are prepared by these successive alkylation procedures utilizing Cl₃SiCH₂Cl as a starting reactant.

To prepare compounds of formula III wherein n is one, the appropriate R₄ R₅ R₆ silyl methyl chloride can be subjected to a displacement reaction by treatment with potassium phthalimide or sodium azide to obtain the corresponding phthalimide or azide. Conversion of the phthalimide to the desired amine can be by reaction with hydrazine hydrate and conversion of the azide can be through chemical reduction to its amine, and subsequent purification of the so-prepared amines may be accomplished via its N-Boc derivative that can then be converted to the amine by hydrolysis.

In effecting the foregoing reaction, the formation of the phthalimide can readily be accomplished by standard reaction conditions for the displacement reaction, preferably by heating the reactants in an inert solvent, e.g., dry dimethylformamide at 70° C. The conversion of the phthalimide to its corresponding amine can be effected by reaction with hydrazine hydrate in a suitable solvent, preferably ethanol, followed by treatment with aqueous acid, preferably HCl, under reflux conditions.

In effecting the foregoing reaction, the formation of the azide can readily be accomplished by standard reaction conditions for the displacement reaction, preferably by heating the reactants in an inert solvent (e.g., dry dimethylformamide) at 40° C. The conversion of the azide to the corresponding amine can be effected through its N-Boc derivative by the sequential treatment with (a) triphenylphosphine (PO₃) about room temperature in tetrahydrofuran (THF) (b) treatment with water followed by (c) purification of the desired product by the formation of its N-t-butoxycarbonyl derivative by reaction with (BOC) O in THF at room temperature. The N-Boc derivative is converted to its amine HCl salt by reaction with gaseous HCl in diethylether (i.e., 3N HCl in diethylether) at room temperature.

To prepare compounds of formula III wherein n is 2, esters derived from the appropriate silylchloride can be reduced to their corresponding alcohols, preferably with lithium aluminum hydride and the alcohols can be converted to their corresponding phthalimides using Mitsunobu reaction conditions (i.e., treatment of the alcohol with diethylazodicarboxylate, triphenyl phosphine and phthalimide). The resulting phthalimides can be hydrolyzed to the corresponding amine hydrochloride by sequential reaction with hydrazine hydrate and aqueous HCl. The esters can be prepared by alkylation of the appropriate silylchloride with a metallo derivative (preferably zinc or sodium) of ethyl acetate according to standard and well-known conditions. Alternatively, compounds may be reacted with magnesium in diethylether to form the appropriate Grignard reagent which, when treated with formaldehyde (preferably using paraformaldehyde), will yield corresponding alcohols.

### Protecting Groups

Oftentimes during the synthesis of complex molecules, one functional group of the molecule interferes with an intended reaction on a second functional group elsewhere in the same molecule. Typically, temporarily masking or “protecting” the more reactive functional group thereby encouraging the desired reaction can circumvent this problem. Protection essentially involves three steps: 1) introducing the protecting group onto the functional group to be protected by means of a protecting group carrying reagent; 2) carrying out the desired reaction; and 3) removing the protecting group.

Protection and deprotection are inevitable requirements of a lengthy synthetic sequence to generate synthetic chemical products, fine chemical intermediates, or important industrial or pharmaceutical organic materials. Accordingly, many protective groups and reagents capable of introducing them into synthetic processes have been and are continuing to be developed today. However, not every molecule can serve as a useful protecting group. Rather, a protecting group must fulfill a number of requirements in order to be useful in carrying out selected syntheses.

First, a protecting group needs to react selectively and be easily attached to the functional group it is supposed to protect. Also, there must be a good yield of the protected compound. Further, a protecting group needs to be resistant to the certain reagents that would otherwise attack the group it protects, and it must not harm the other functional groups in the molecule. In other words, the protected compound needs to remain stable as it proceeds through the multiple steps in the synthetic sequence. Finally, a protecting group needs to be easily removed under conditions that will not adversely react with the regenerated functional group.

Protecting groups exist for various functional moieties and have their own pattern of chemoselectivity during deprotection. In molecules with multiple discrete simultaneous protections, a careful strategy exists for specific removal and modification of the exposed functionality. Thus, elaborately protected, highly functional templates can serve as total synthetic intermediates.

To date, a remarkable variety of protecting reagents has been reported, and the preparation of the reagents as well as the protection and deprotection strategies under a variety of conditions have been summarized in the literature. In addition, as should be appreciated, more elaborate syntheses cannot be accomplished with only a few protecting groups. Rather, such elaborate syntheses can typically only succeed with the use of a large number of mutually complementary protecting groups. Accordingly, great strides have been made to synthesize new protecting groups that complement existing protecting groups.

Examples of protecting groups and the corresponding reagents can be found in Green et al., “Protective Groups in Organic Chemistry”, (Wiley, 2. sup.nd ed. 1991) and Harrison et al., “Compendium of Synthetic Organic Methods”, Vols. 1-8 (John Wiley and Sons, 1971-1996). For example, protecting groups have been developed for the protection of hydroxy groups, amine groups, carbonyl groups, and carboxyl groups and thiols, to name a few.

In certain methods, the final step utilized in the preparation of the compounds of formula I entails the removal of N-protecting and S-protecting groups to form the free amine/free thiol and/or pharmaceutically acceptable salts thereof. Other equivalently functioning protecting groups are known and may also be utilized and are contemplated.

### General Procedure for Amidation of Carboxylic Acids

Following secondary amine and thiol protection, carboxylic acid sulfhydryl compounds are converted to the corresponding amides. Several methods for accomplishing this conversion are known and can be used. For example, a dichlomethane solution of carboxylic acid sulfhydryl compounds is allowed to cool to −5° C. in a salted ice-bath. To the cooled solution, triethylamine is added followed by addition of ethyl chloroformate. In addition to ethyl chloroformate a variety of other compounds can be used including thionyl chloride, phosphorous chloride and oxalyl chloride. The reaction mixture can be stirred for 15 min., then, an appropriate silyl amine derivative of formula III can be added dropwise, and the reaction allowed to proceed at −5° C. for 25 min and further, for overnight at room temperature. The reaction mixture can then be washed with 5% hydrochloric acid, then with sodium bicarbonate solution and finally with water. The dichloromethane solution can then be dried over magnesium sulfate and evaporated to dryness. Finally, a solution of sulfhydryl compound amides in 2M HCl methanolic solution can be stirred at room temperature for 24 h. The dichloromethane solution can then be evaporated. The silyl amide derivative products can be recrystallized from an appropriate solvent.

Compounds of the invention may be chiral. They may be in the form of a single enantiomer or diastereomer, or a racemate. The stereochemistry of a chiral ring atom is preferably the same as that of the corresponding atom in the parent analog. More preferably, the stereochemistry of the compound as a whole corresponds to that of the parent molecule.

Compounds of the invention can be prepared in racemic form, or prepared in individual enantiomeric form by specific synthesis or resolution. The compounds may, for example, be resolved into their enantiomers by standard techniques, such as the formation of diastereomeric pairs by salt formation with an optically active acid followed by fractional crystallization and regeneration of the free base. Alternatively, the enantiomers of the novel compounds may be chromatographically separated, such as by HPLC, for example by using a chiral column

Some compounds may exist in the form of various solvates, such as hydrates and also fall within the scope of the present invention.

Compounds of the invention may be in the form of pharmaceutically acceptable salts, for example, addition salts of inorganic or organic acids. Such inorganic acid addition salts include, for example, salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl)benzoicacid, 1-hydroxy-2-naphthoicacid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulpuric acid, mucic acid, 2-naphthalenesulphonic acid, pamoic acid, pantothenic acid, phosphanilic acid ((4-aminophenyl)phosphonic acid), picric acid, salicylic acid, stearic acid, succinic acid, tannic acid, tartaric acid, terephthalic acid, p-toluenesulphonic acid, 10-undecenoic acid and the like.

It will be appreciated that such salts, provided that they are pharmaceutically acceptable, may be used in therapy. Such salts may be prepared by reacting the compound with a suitable acid in a conventional manner.

A compound of the invention may be prepared by any suitable method known in the art. Mixtures of final products or intermediates obtained can be separated on the basis of the physical-chemical differences of the constituents, by known methods, into the pure final products or intermediates, for example by chromatography, distillation, fractional crystallization, or by formation of a salt if appropriate or possible under the circumstances.

The preparation of the compounds of formula I are affected by a variety of procedures depending primarily upon the specific definitions of the n and R₁, R₂, R₃ R₄, R₅ and R₆ moieties. One of skill can appreciate that chemical reactions and procedures analogous to those known in the art and the selection of a particular route to obtain particular compounds is governed by known principles and can be obtained using methods that are analogous to the disclosed methods.

Preferred silicon derivatives that can be generated using such methods include the following:
(R)-2-acetamido-3-mercapto-N-((trimethylsilyl)methyl)propanamide,
(R)-2-acetamido-3-mercapto-N-(3-(trimethylsilyl)propyl)propanamide,
(R)-2-acetamido-N-((dimethyl(propyl)silyl)methyl)-3-mercaptopropanamide,
(R)-2-acetamido-3-mercapto-N-(4-(trimethylsilyl)butyl)propanamide,
(R)-2-acetamido-N-((butyldimethylsilyl)methyl)-3-mercaptopropanamide,
(R)-2-acetamido-3-mercapto-N-(5-(trimethylsilyl)pentyl)propanamide,
(R)-2-acetamido-N-((dimethyl(pentyl)silyl)methyl)-3-mercaptopropanamide,
(R)-2-acetamido-3-mercapto-N-(6-(trimethylsilyl)hexyl)propanamide,
(R)-2-acetamido-N-((hexyldimethylsilyl)methyl)-3-mercaptopropanamide,
(R)-2-acetamido-3-mercapto-N-(7-(trimethylsilyl)heptyl)propanamide,
(R)-2-acetamido-N-((heptyldimethylsilyl)methyl)-3-mercaptopropanamide,
(2R)-2-acetamido-N-(1,1-dimethylsilinan-3-yl)-3-mercaptopropanamide,
(R)-2-acetamido-3-mercapto-N-(4-(trimethylsilyl)phenyl)propanamide,
(R)—N-(4-(trimethylsilyl)benzyl)-2-acetamido-3-mercaptopropanamide
(R)-2-acetamido-3-mercapto-N-(4-((trimethylsilyl)methyl)phenyl)propanamide
(R)-2-acetamido-3-mercapto-N-(6-(trimethylsilyl)pyridin-3-yl)propanamide,
(R)-2-acetamido-N-((dimethyl(pyridin-3-yl)silyl)methyl)-3-mercaptopropanamide,
(R)-2-acetamido-N-(2-(dimethyl(pyridin-3-yl)silyl)ethyl)-3-mercaptopropanamide,
(R)-2-acetamido-N-((dimethyl(phenyl)silyl)methyl)-3-mercaptopropanamide,
(R)-2-acetamido-N-(((4-fluorophenyl)dimethylsilyl)methyl)-3-mercaptopropanamide,
(R)-2-acetamido-N-(((4-chlorophenyl)dimethylsilyl)methyl)-3-mercaptopropanamide,
(R)-2-acetamido-3-mercapto-N-(((4-methoxyphenyl)dimethylsilyl)methyl)propanamide,
(R)-2-acetamido-N-(2-(dimethyl(phenyl)silyl)ethyl)-3-mercaptopropanamide,
(R)-2-acetamido-N-(2-((4-fluorophenyl)dimethylsilyl)ethyl)-3-mercaptopropanamide,
(R)-2-acetamido-N-(2-((4-chlorophenyl)dimethylsilyl)ethyl)-3-mercaptopropanamide,
(R)-2-acetamido-3-mercapto-N-(2-((4-methoxyphenyl)dimethylsilyl)ethyl)propanamide and

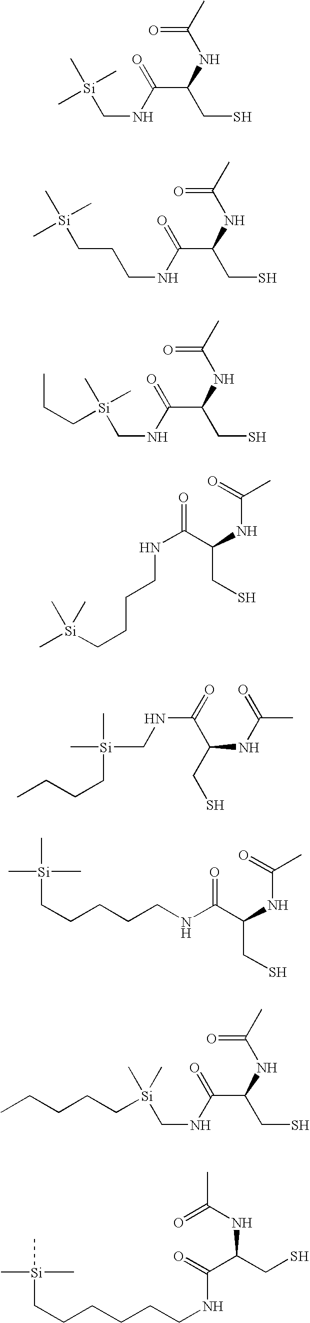

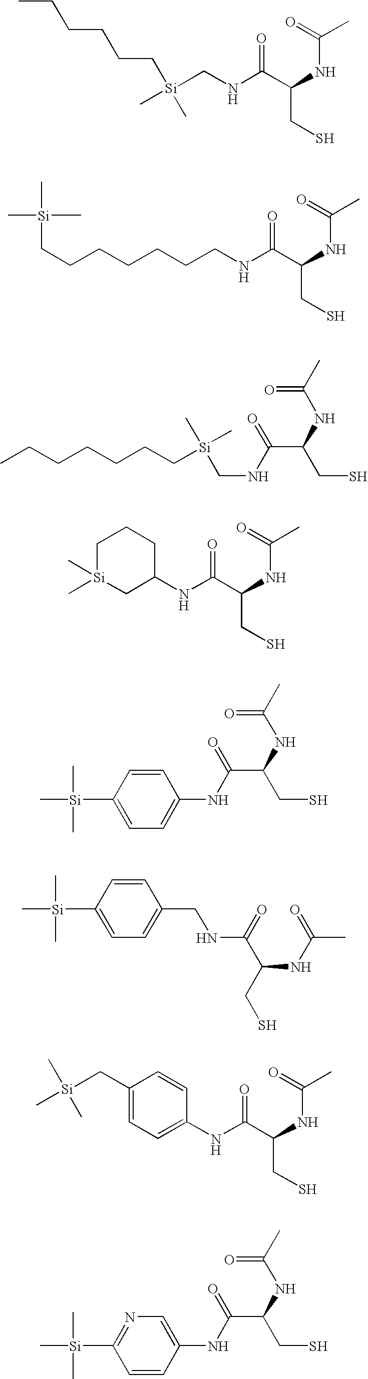

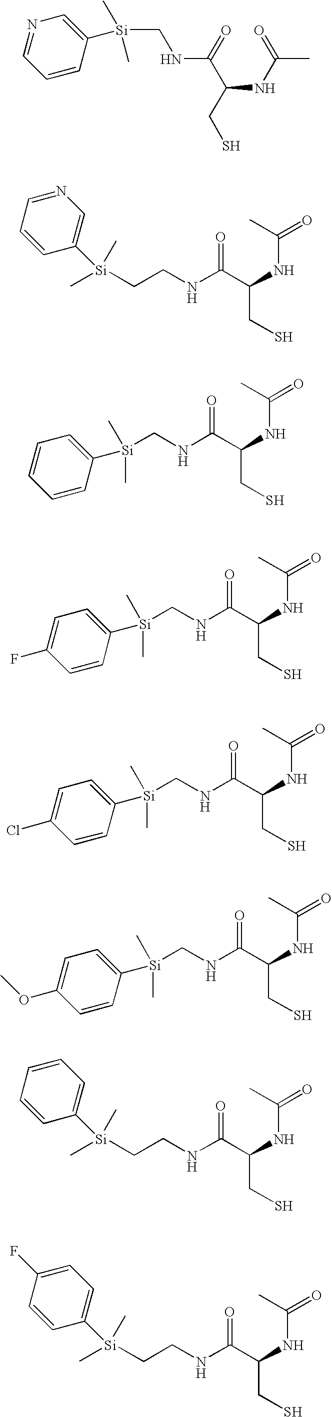

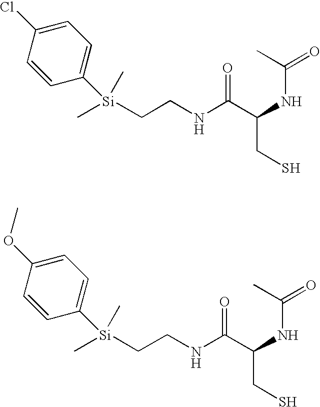

Treatment is contemplated in mammals, particularly humans, as well as those mammals of economic or social importance, or of an endangered status. Examples may be livestock or other animals expressly for human consumption, or domesticated animals such as dogs, cats, or horses. Also contemplated is the treatment of birds and poultry, such as turkeys, chickens, and fowl of the like.

Determination of a therapeutically effective amount is well within the capability of those skilled in the art, especially in light of the detailed disclosure provided herein.

Toxicity and therapeutic efficacy of the compounds described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition.

Depending on the severity and responsiveness of the condition to be treated, dosing can also be a single administration of a slow release composition, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present invention can be used to treat any one of a plurality of diseases, disorders or conditions associated with the formation of oxidative stress.

As used herein, the term “treat” includes substantially inhibiting, slowing or reversing the progression of a disease, disorder or condition, substantially ameliorating clinical symptoms of a disease disorder or condition, or substantially preventing the appearance of clinical symptoms of a disease, disorder or condition.

The compounds of the present invention can be used to treat non-central nervous system disorders such as rheumatoid arthritis, cataract, Down syndrome, cystic fibrosis, diabetes, acute respiratory distress syndrome, asthma, post-surgical neurological dysfunction, amyotrophic lateral sclerosis, atherosclerotic cardiovascular disease, hypertension, post-operative restenosis, pathogenic vascular smooth muscle cell proliferation, pathogenic intra-vascular macrophage adhesion, pathogenic platelet activation, pathogenic lipid peroxidation, myocarditis, stroke, multiple organ dysfunction, complication resulting from inflammatory processes, AIDS, cancer, aging, bacterial infection, sepsis; viral disease, such as AIDS, hepatitis C, an influenza and a neurological viral disease, all of which were previously shown to be associated with the formation and/or overproduction of oxidants.

The compounds of the present invention can also be used to treat a central nervous system disorder characterized by oxidative stress, such as, neurodegenerative disorders, Parkinson's disease, Alzheimer's disease, Creutzfeldt-Jakob disease, cerebral ischemia, multiple sclerosis, degenerative diseases of the basal ganglia, motoneuron diseases, scrapies, spongiform encephalopathy, neurological viral diseases, motoneuron diseases, post-surgical neurological dysfunction and loss or memory impairment including chemobrain, all of which were previously shown to be associated with the formation and/or overproduction of oxidants.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above descriptions illustrate the invention in a non-limiting fashion.

### Example

### (R)-2-acetamido-3-mercapto-N-(3-(trimethylsilyl)propyl)propanamide

Chemical formula: C₁₁H₂₄N₂O₂SSi: (molecular weight of 276.47): C, 47.79; H, 8.75; N, 10.13; O, 11.57; S, 11.60; Si, 10.16. (R)-4-carboxy-3-acetyl-2,2-dimethylthiazolidine. A suspension of N-acetyl-R-cysteine (1.0 g, 0.006 mol) and montmorillonite K10 (0.2 g, 20 wt. %) in 40 mL of anhydrous acetone/2,2-dimethoxypropane (1:3) mixture was stirred at room temperature for 3 h. The reaction mixture was then filtered, and solvent was evaporated to give (R)-4-carboxy-3-acetyl-2,2-dimethylthiazolidine (1.03 g, 84% yield) as a white solid (90% pure by 1H NMR spectroscopy), which was used for the next step without further purification.

(R)-4-(trimethylsilyl)propyl)amide-3-acetyl-2,2-dimethylthiazolidine. A solution of (R)-4-carboxy-3-acetyl-2,2-dimethylthiazolidine (1.03 g, 0.005 mol) and triethylamine (0.7 mL, 0.005 mol) in 20 mL of dichloromethane was cooled to −5° C. and a solution of ethyl chloroformate (0.5 mL, 0.005 mol) in 5 mL of dichloromethane was added dropwise. After 15 minutes of stirring at −5° C., to the reaction mixture trimethylsilylpropylamine (1.2 mL, 0.005 mol) was slowly added. Stirring was continued for 25 minutes at −5° C. and 15 h at room temperature. The reaction mixture was then diluted with 30 mL of dichloromethane and washed thoroughly with 30 mL portions of 5% hydrochloric acid, then with sodium bicarbonate solution and finally with water. The dichloromethane solution was then dried over magnesium sulfate and evaporated to give (R)-4-(trimethylsilyl)propyl)amide-3-acetyl-2,2-dimethylthiazolidine (1.2 g, 59% yield) as a pale yellow oil, 85% pure by 1H NMR spectroscopy, which was used for the next step without further purification.

(R)-2-acetamido-3-mercapto-N-(3-(trimethylsilyl)propyl)propanamide. A solution of (R)-4-(trimethylsilyl)propyl)amide-3-acetyl-2,2-dimethylthiazolidine (0.8 g, 0.002 mol) in 30 mL of 2M HCl methanolic solution was stirred at room temperature for 24 h. The dichloromethane solution was then evaporated to give (R)-2-acetamido-3-mercapto-N-(3-(trimethylsilyl)propyl)propanamide (0.5 g, 67% yield) as a pale yellow oil (85% pure by 1H NMR spectroscopy). Recrystallization from dichloromethane/hexane mixture at −20° C. gave pure product (0.4 g, 57% yield) as a white solid.

This methodology was used to successfully synthesize (R)-2-acetamido-3-mercapto-N-((trimethylsilyl)methyl)propanamide (Example 2) and (R)-2-acetamido-N-((dimethyl(phenyl)silyl)methyl)-3-mercaptopropanamide (Example 3). These example compounds were evaluated for in vitro anti-cancer activity compared to N-acetyl-L-cysteine.

CellTiter-Blue® Reagent Method: Following incubation with test compounds for 72 or 96 hours, human MD-MBA-231 breast cancer cells were briefly washed, fixed and stained with the CellTiter-Blue® Reagent resazurin. Resazurin measures the metabolic capacity of cells, an indicator of cell viability. Viable cells retain the ability to reduce resazurin into resorufin, that is highly fluorescent. Nonviable cells rapidly lose metabolic capacity, do not reduce the indicator dye, and therefore do not generate a fluorescent signal. This test measures the degree of cytotoxicity caused by the test material.

IC₅₀ determination: Data are expressed as the percentage of survival of the untreated (vehicle) control calculated from the fluorescence corrected for background absorbance. The surviving fraction of cells were determined by dividing the mean fluorescence values of the test agents by the mean fluorescence values of untreated control. The inhibitory concentration value for the test agent(s) and control were estimated using Prism 4 software (GraphPad Software, Inc.) by curve-fitting the data using the non-linear regression analysis. In some instances IC50 values could not be extrapolated and these are marked (*). Compounds of the invention were tested as monotherapy and results presented in TABLE 1

TABLE 1
Monotherapy

| | 72 Hours | 96 Hours |
|---|---|---|
| | Treatment | Treatment |
| COMPOUND | IC50 (μM) | IC50 (μM) |
| N-Acetyl-L-Cysteine | 277.40 | * |
| Eample 1 | 21.62 | 18.83 |
| (R)-2-acetamido-3-mercapto-N-(3- | | |
| (trimethylsilyl)propyl)propanamide | | |
| Example 2 | 131.50 | 136.50 |
| (R)-2-acetamido-3-mercapto-N- | | |
| ((trimethylsilyl)methyl)propanamide | | |
| Example 3 | 28.08 | 24.33 |
| (R)-2-acetamido-N- | | |
| ((dimethyl(phenyl)silyl)methyl)-3- | | |
| mercaptopropanamide | | |

Compounds of the invention were tested in combination with inhibitory concentration 30% and 10% (IC₃₀/IC₁₀) of Adriamycin (doxorubicin) and results presented in TABLE 2

TABLE 2
Concurrent combination treatment

| | 72 Hours IC₁₀ | 72 Hours IC₃₀ | 96 Hours IC₁₀ | 96 Hours IC₃₀ |
|---|---|---|---|---|
| | Doxorubicin | Doxorubicin | Doxorubicin | Doxorubicin |
| COMPOUND | (μM) | (μM) | (μM) | (μM) |
| N-Acetyl-L-Cysteine | 129.70 | 639.30 | * | 925.2 |
| Eample 1 | 7.49 | 29.67 | 6.703 | 26.70 |
| (R)-2-acetamido-3-mercapto-N-(3- | | | | |
| (trimethylsilyl)propyl)propanamide | | | | |
| Example 2 | 49.42 | 269.0 | 34.55 | * |
| (R)-2-acetamido-3-mercapto-N- | | | | |
| ((trimethylsilyl)methyl)propanamide | | | | |
| Example 3 | 8.553 | 39.29 | 9.479 | 30.63 |
| (R)-2-acetamido-N- | | | | |
| ((dimethyl(phenyl)silyl)methyl)-3- | | | | |
| mercaptopropanamide | | | | |

Compounds of the invention were tested in combination with inhibitory concentration 30% and 10% (IC₃₀/IC₁₀) of Adriamycin (doxorubicin) following 6 hours of pretreatment with example compounds and results presented in TABLE 3

TABLE 3
6-hour Pretreatment before doxorubicin administration

| | 72 Hours IC₁₀ | 72 Hours IC₃₀ | 96 Hours IC₁₀ | 96 Hours IC₃₀ |
|---|---|---|---|---|
| | Doxorubicin | Doxorubicin | Doxorubicin | Doxorubicin |
| COMPOUND | (μM) | (μM) | (μM) | (μM) |
| N-Acetyl-L-Cysteine | 499.10 | * | 454.10 | * |
| Eample 1 | 27.13 | 10.04 | 34.20 | 2.162 |
| (R)-2-acetamido-3-mercapto-N-(3- | | | | |
| (trimethylsilyl)propyl)propanamide | | | | |
| Example 2 | 210.70 | 56.64 | * | 14.68 |
| (R)-2-acetamido-3-mercapto-N- | | | | |
| ((trimethylsilyl)methyl)propanamide | | | | |
| Example 3 | 35.69 | 11.21 | 37.88 | 3.799 |
| (R)-2-acetamido-N- | | | | |
| ((dimethyl(phenyl)silyl)methyl)-3- | | | | |
| mercaptopropanamide | | | | |

The foregoing descriptions have been directed to particular embodiments of the invention in accordance with requirements of the Patent Statutes for the purposes of illustration and explanation. It will be apparent however, to those skilled in the art, that many modifications, changes and variations in the claimed compositions, solutions, methods of administration of the compositions set forth will be possible without departing from the scope and spirit of the claimed invention. It is intended that the following claims be interpreted to embrace all such modifications and changes.

1. A compound of formula (I), 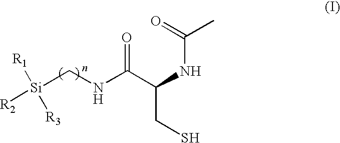
wherein
n is an integer;
and wherein R₁, R₂, R₃ can be the same or different and can be selected from the chemical groups consisting of methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and hydrates and solvates thereof.

2. A compound according to claim 1, selected from a group consisting of:
(R)-2-acetamido-3-mercapto-N-((trimethylsilyl)methyl)propanamide,
(R)-2-acetamido-3-mercapto-N-(3-(trimethylsilyl)propyl)propanamide,
(R)-2-acetamido-N-((dimethyl(propyl)silyl)methyl)-3-mercaptopropanamide,
(R)-2-acetamido-3-mercapto-N-(4-(trimethylsilyl)butyl)propanamide
(R)-2-acetamido-N-((butyldimethylsilyl)methyl)-3-mercaptopropanamide,
(R)-2-acetamido-3-mercapto-N-(5-(trimethylsilyl)pentyl)propanamide,
(R)-2-acetamido-N-((dimethyl(pentyl)silyl)methyl)-3-mercaptopropanamide,
(R)-2-acetamido-3-mercapto-N-(6-(trimethylsilyl)hexyl)propanamide,
(R)-2-acetamido-N-((hexyldimethylsilyl)methyl)-3-mercaptopropanamide,
(R)-2-acetamido-3-mercapto-N-(7-(trimethylsilyl)heptyl)propanamide,
(R)-2-acetamido-N-((heptyldimethylsilyl)methyl)-3-mercaptopropanamide,
(2R)-2-acetamido-N-(1,1-dimethylsilinan-3-yl)-3-mercaptopropanamide,
(R)-2-acetamido-3-mercapto-N-(4-(trimethylsilyl)phenyl)propanamide,
(R)—N-(4(trimethylsily)benzyl)-2-acetamido-3-mercaptopropanamide,
(R)-2-acetamido-3-mercapto-N-(4-((trimethylsilyl)methyl)phenyl)propanamide,
(R)-2-acetamido-3-mercapto-N-(6-(trimethylsilyl)pyridin-3-yl)propanamide,
(R)-2-acetamido-N-((dimethyl(pyridin-3-yl)silyl)methyl)-3-mercaptopropanamide,
(R)-2-acetamido-N-(2-(dimethyl(pyridin-3-yl)silyl)ethyl)-3-mercaptopropanamide,
(R)-2-acetamido-N-((dimethyl(phenyl)silyl)methyl)-3-mercaptopropanamide,
(R)-2-acetamido-N-(((4-fluorophenyl)dimethylsilyl)methyl)-3-mercaptopropanamide,
(R)-2-acetamido-N-(((4-chlorophenyl)dimethylsilyl)methyl)-3-mercaptopropanamide,
(R)-2-acetamido-3-mercapto-N-(((4-methoxyphenyl)dimethylsilyl)methyl)propanamide,
(R)-2-acetamido-N-(2-(dimethyl(phenyl)silyl)ethyl)-3-mercaptopropanamide,
(R)-2-acetamido-N-(2-((4-fluorophenyl)dimethylsilyl)ethyl)-3-mercaptopropanamide,
(R)-2-acetamido-N-(2-((4-chlorophenyl)dimethylsilyl)ethyl)-3-mercaptopropanamide and
(R)-2-acetamido-3-mercapto-N-(2-((4-methoxyphenyl)dimethylsilyl)ethyl)propanamide

3. A pharmaceutical composition comprising a therapeutically effective amount of one or more compounds of formula (I) as an active agent 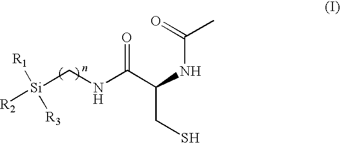
wherein
n is an integer;
and wherein Ri, R₂, R₃ can be the same or different and can include a group selected from the groups consisting of methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, P—(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl, and hydrates and solvates thereof; and an inert carrier.

4. The pharmaceutical composition of claim 3, further comprising a second distinct active agent.

5. The compound of claim 1, wherein the compound is a diasteriomer, racemate, single enantiomer.

6. The compound of claim 1, wherein the compound is in a hydrated form, solvated form, polymorphic form, crystalline form or an amorphous form.

7. The composition of claim 1 wherein n is 1-6.

8. A method for the preparation of a compound of formula I which comprises:
(a) reacting a compound of formula II to generate a compound of formula Iia 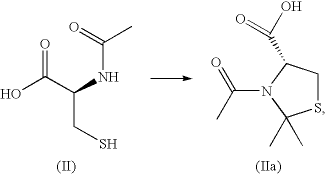
(b) converting the compound of formula Iia to an acid chloride (formula Iib), 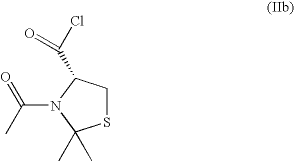
(c) reacting a compound of formula lib with a compound of formula III 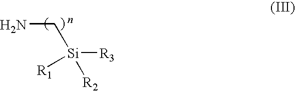
wherein
n is an integer;
and wherein R₁, R₂, R₃ can be the same or different and can include a group selected from the groups consisting of methyl, ethyl, n-propyl, iso-propyl, n-butyl, iso-butyl, sec-butyl, tert-butyl, —CH₂CH(CH₂CH₃)₂, 2-methyl-n-butyl, 6-fluoro-n-hexyl, phenyl, benzyl, cyclohexyl, cyclopentyl, cycloheptyl, allyl, iso-but-2-enyl, 3-methylpentyl, —CH₂-cyclopropyl, —CH₂-cyclohexyl, —CH₂CH₂-cyclopropyl, —CH₂CH₂-cyclohexyl, —CH₂-indol-3-yl, p-(phenyl)phenyl, o-fluorophenyl, m-fluorophenyl, p-fluorophenyl, m-methoxyphenyl, p-methoxyphenyl, phenethyl, benzyl, m-hydroxybenzyl, p-hydroxybenzyl, p-nitrobenzyl, m-trifluoromethylphenyl, p-(CH₃)₂NCH₂CH₂CH₂O-benzyl, p-(CH₃)₃COC(O)CH₂O-benzyl, p-(HOOCCH₂O)-benzyl, 2-aminopyrid-6-yl, p-(N-morpholino-CH₂CH₂O)-benzyl, —CH₂CH₂C(O)NH₂, —CH₂-imidazol-4-yl, —CH₂-(3-tetrahydrofuranyl), —CH₂-thiophen-2-yl, —CH₂ (1-methyl)cyclopropyl, —CH₂-thiophen-3-yl, thiophen-3-yl, thiophen-2-yl, —CH₂—C(O)O-t-butyl, —CH₂—C(CH₃)₃, —CH₂CH(CH₂CH₃)₂, -2-methylcyclopentyl, -cyclohex-2-enyl, —CH[CH(CH₃)₂]COOCH₃, —CH₂CH₂N(CH₃)₂, —CH₂C(CH₃)═CH₂, —CH₂CH═CHCH₃ (cis and trans), —CH₂OH, —CH(OH)CH₃, —CH(O-t-butyl)CH₃, —CH₂OCH₃, —(CH₂)₄NH-Boc, —(CH₂)₄NH₂, —CH₂-pyridyl (e.g., 2-pyridyl, 3-pyridyl and 4-pyridyl), pyridyl (2-pyridyl, 3-pyridyl and 4-pyridyl), —CH₂-naphthyl (e.g., 1-naphthyl and 2-naphthyl), —CH₂—(N-morpholino), p-(N-morpholino-CH₂CH₂O)-benzyl, benzo[b]thiophen-2-yl, 5-chlorobenzo[b]thiophen-2-yl, 4,5,6,7-tetrahydrobenzo[b]thiophen-2-yl, benzo[b]thiophen-3-yl, 5-chlorobenzo[b]thiophen-3-yl, benzo[b]thiophen-5-yl, 6-methoxynaphth-2-yl, —CH₂CH₂ SCH₃, thien-2-yl, thien-3-yl; and (d) reacting the product of step (c) with an acid and purifying a silyl amide derivative.

9. A method for treatment of a subject having a condition or disorder associated with formation of oxidative stress that comprises administering a therapeutically effective amount of a compound of formula 1 or a pharmaceutically acceptable salt, solvate or polymorph thereof.

10. The method of claim 9, in which the condition or disorder is angiogenesis.

11. The method of claim 9, in which the condition or disorder is cancer.

12. The method of claim 9, wherein the condition or disorder associated with formation of oxidative stress is a central nervous system disease.

13. The method of claim 12, wherein said central nervous system disease is selected from the group comprising a neurodegenerative disorder, Parkinson's disease, Alzheimer's disease, Creutzfeldt-Jakob disease, cerebral ischemia, multiple sclerosis, a degenerative disease of the basal ganglia, a motoneuron disease, scrapies, spongiform encephalopathy, a neurological viral disease, a motoneuron disease, post-surgical neurological dysfunction, memory loss, chemobrain and memory impairment.

14. The method of claim 9, wherein the condition associated with formation of oxidative stress is a non-central nervous system disease.

15. The method of claim 14, wherein said non-central nervous system disease is selected from the group comprising rheumatoid arthritis, cataract, Down's syndrome, cystic fibrosis, diabetes, acute respiratory distress syndrome, asthma, post-surgical neurological dysfunction, amyotrophic lateral sclerosis, atherosclerotic cardiovascular disease, hypertension, post-operative restenosis, pathogenic vascular smooth muscle cell proliferation, pathogenic intra-vascular macrophage adhesion, pathogenic platelet activation, pathogenic lipid peroxidation, myocarditis, stroke, multiple organ dysfunction, complication resulting from inflammatory processes, AIDS, aging, bacterial infection, sepsis; viral disease, AIDS, hepatitis C, influenza and a neurological viral disease.

16. The method of claim 9, wherein the subject is a mammal.

17. The method of claim 9, wherein the subject is human.

18. The method of claim 9, wherein the compound is administered in a pharmaceutical composition that includes a pharmaceutically acceptable carrier.

19. The method of claim 9, wherein said administering step is by intranasal, transdermal, intradermal, oral, buccal, parenteral, topical, rectal or inhalation administration.

20. The method of claim 9, wherein the composition further includes a formulating agent selected from the group consisting of a suspending agent, a stabilizing agent and a dispersing agent.

21. The method of claim 9, wherein a therapeutically effective amount of a compound of formula 1 or a pharmaceutically acceptable salt, solvate or polymorph thereof is administered in advance of or concurrently with an antineoplastic compound selected from the group consisting of antibiotic agents, alkylating agents, antimetabolite agents, hormonal agents, immunological agents, interferon agents, wherein: the amount of conjunctive therapy and the amount of the compound of the invention together comprise a neoplasia-treating-effective amount; and the neoplasia is sensitive to such treatment.

22. The method of claim 9, wherein a therapeutically effective amount of a compound of formula 1 or a pharmaceutically acceptable salt, solvate or polymorph thereof is administered for the prevention or treatment of toxicities due to chemotherapeutic drug treatment.

23. A method for treating a neoplasia in a subject in need of such treatment wherein a therapeutically effective amount of a compound of formula 1 or a pharmaceutically acceptable salt, solvate or polymorph thereof is administered with ionizing radiation: the amount of radiation and the amount of the compound of formula 1 together comprise a neoplasia-treating-effective amount such that the neoplasia is sensitive to the treatment.

24. The method of claim 23, wherein a therapeutically effective amount of a compound of formula 1 or a pharmaceutically acceptable salt, solvate or polymorph thereof is administered for the prevention or treatment of toxicities due to radiation (radiotherapy), in particular whole brain radiation.
